(19) United States
(12) Patent Application Publication
(10) Pub. No.: US 2019/0152993 A1
(43) Pub. Date: May 23, 2019

(54) Reversibly Switchable Surfactants and Methods of Use

(71) Applicant: WARNER BABCOCK INSTITUTE FOR GREEN CHEMISTRY, LLC, Wilmington, MA (US)

(72) Inventors: John C. Warner, Wilmington, MA (US); Srinivasa R. Cheruku, Lexington, MA (US)

(73) Assignee: WARNER BABCOCK INSTITUTE FOR GREEN CHEMISTRY, LLC, Wilmington, MA (US)

(21) Appl. No.: 16/313,312

(22) PCT Filed: Jun. 23, 2017
(86) PCT No.: PCT/US2017/038954

§ 371 Date: Dec. 26, 2018

Related U.S. Application Data
(60) Provisional application No. 62/353,805, filed on Jun. 23, 2016.

(51) Int. Cl. C07D 513/10; C07D 498/10; C07D 491/107; C09D 7/63; C02F 1/44

(52) U.S. Cl. CPC C07D 513/10; C07D 498/10; C02F 2103/08; C09D 7/63; C02F 1/445; C07D 491/107

(57) ABSTRACT

Disclosed herein are reversibly-switchable surfactants and methods of extracting natural products, coating surfaces, cleaning laundry, and osmotic extraction using same.

### BACKGROUND

Surfactants are amphiphilic compounds comprised of a polar or ionic “head” group and a non-polar hydrophobic “tail,” typically a hydrocarbon chain. Surfactants may serve as dispersants, emulsifying agents, detergents, wetting agents, and/or foaming agents. Surfactants are also used in synthesis, such as emulsion polymerization, to compartmentalize or stabilize reactants and/or products to control size, morphology or other properties.

Though surfactants are widely used in a variety of processes, it is rare that the surfactant itself is a desirable component of the final product. Removal of surfactants often requires heat, mechanical agitation, and or additional chemical modification with some form of neutralizing agent. Such treatments can be costly, and can lead to product contamination and produce large volumes of contaminated aqueous wastes. An alternative is to use biodegradable surfactants, which are disposed of with aqueous waste. Preferably, the biodegradation is triggered by some controllable means. While using this type of surfactant lowers remediation costs and may be less energy-intensive than traditional surfactant removal, it still poses concern in regard to water-borne pollution.

Cleavable surfactants are molecules that, when triggered by ozone, UV light, heat, and/or a change in pH, lose their surfactant character by cleavage of the polar head of the surfactant from the non-polar tail. A disadvantage of cleavable surfactants is that the non-polar cleaved head and the polar tail have substantively different solubilities, so that the head and tail partition themselves such that one of them is in each of the two separated phases. This can lead to contamination of both separated phases, creating waste solvent and contamination of the isolated solute or secondary phase.

Another class of triggered reagents comprises reversibly switchable surfactants. Like most surfactants, these materials facilitate reactions and/or mixing by contacting immiscible materials or overcoming transport limitations. Similar to cleavable surfactants, reversibly switchable surfactants are controlled by a switch that suppresses or eliminates surfactant activity. However, these switches are also reversible, where either the surfactant decomposition products are reconstituted into the original surfactant for reuse, or the surfactant does not decompose at all, but rather simply changes form in a reversible process. Examples of reversible triggers or switches include carbon dioxide, air, oxidation/reduction cycles, and photochemistry.

Switching the surfactant from active to de-activated forms enables the separation of the surfactant from a reagent, eliminates the need for two solvent phases, and provides for re-use of the surfactant and solvent, thereby saving energy and reducing waste material and resulting cost and environmental concerns.

### SUMMARY

The present application discloses a photochromic, reversibly-switchable surfactant comprising a spiropyran head group and a hydrophobic tail. The spiropyran head group can be reversibly switched between the ground state (hydrophobic, charge-neutral spiropyran) and an activated state (hydrophilic, zwitterionic merocyanine). The hydrophobic tail is not switchable. In the activated merocyanine form, the molecule functions as a surfactant. Triggering the reversion to the ground state spiropyran form causes de-activation of the hydrophilic character of the head group and loss of the surfactant properties. The surfactant remains a single intact molecule in both the ground state and active forms.

Methods of use of the reversibly-switchable surfactant are described for processes including natural product extraction and isolation, deposition of thin-films and coatings, multi-step chemical synthesis or processing, cleaning laundry and osmotic processes.

Accordingly, described herein is a photochromic, reversibly-switchable surfactant comprising the formula I:

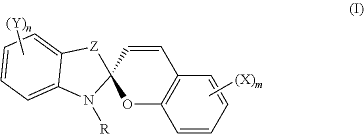

wherein: m is 0, 1, 2, 3 or 4; n is 0, 1, 2, 3 or 4; R is H or is selected from the group consisting of substituted or unsubstituted C₁₋₂₂ alkyl, substituted or unsubstituted C₆₋₁₀ aryl, substituted or unsubstituted —C₁₋₆ alkyl-C₆₋₁₀ aryl, substituted or unsubstituted C₁₋₂₂ alkylC(O)—, substituted or unsubstituted C₁₋₂₂ alkylS(O)₁₋₂—, substituted or unsubstituted C₁₋₂₂ alkylNR′C(O)— and substituted or unsubstituted C₁₋₂₂ alkoxyC(NR″)—, or a polymer, bead, or resin; each X is independently H or is selected from the group consisting of halo, —CN, —NO₂, —COOH, —SH, —OH, substituted or unsubstituted C₁₋₆ alkyl, substituted or unsubstituted C₆₋₁₀ aryl, substituted or unsubstituted —C₁₋₆ alkyl-C₆₋₁₀ aryl, substituted or unsubstituted —C₁₋₆ alkylC(O)—, substituted or unsubstituted —C₁₋₆ alkylS(O)₁₋₂—, substituted or unsubstituted —C₁₋₆ alkylNR′C(O)— and substituted or unsubstituted C₁₋₆ alkoxyC(NR″)— or a polymer; each Y is independently H or is selected from the group consisting of halo, —CN, —NO₂, —COOH, —SH, —OH, substituted or unsubstituted C₁₋₆ alkyl, substituted or unsubstituted C₅₋₁₀ aryl, substituted or unsubstituted —C₁₋₆ alkyl-C₆₋₁₀ aryl, substituted or unsubstituted C₁₋₆ alkylC(O)—, substituted or unsubstituted C₁₋₆ alkylS(O)₁₋₂—, substituted or unsubstituted C₁₋₆ alkylNR′C(O)— and substituted or unsubstituted C₁₋₆ alkoxyC(NR″)— or a polymer; Z is —O—, —S—, —C(O)—, —C(S)—, —C(NR′)—, —S(O)₁₋₂—, —NR′ and —CR′R″—; and R′ and R″ are each independently selected from the group consisting of H, substituted or unsubstituted C₁₋₆ alkyl and substituted and unsubstituted —C₁₋₆ alkyl-C₆₋₁₀ aryl.

Also described herein is a method of coating a surface with a thin-film material comprising the steps of: (a) providing a solution of thin-film material in a solvent in the presence of a switchable surfactant; (b) causing the surfactant to enter the activated state, wherein the activated surfactant solubilizes the thin-film material; (c) coating the surface with the solution; (d) causing the surfactant to switch to the deactivated state, wherein the deactivated surfactant is soluble in the solvent but does not solubilize the thin-film material; and (e) physically removing the solution from the surface.

Also described herein is a method of extracting a natural product from its source material, the method comprising the steps of: (a) providing an admixture of source material in a solvent in the presence of a switchable surfactant; (b) causing the surfactant to enter an activated state, wherein the activated surfactant solubilizes the natural product in the solvent; (c) physically separating the source material from the solvent; (d) causing the surfactant to return to the deactivated state, wherein the deactivated surfactant is soluble in the solvent but does not solubilize the natural product; and (e) physically separating the natural product from the solvent.

Further described herein is a method of cleaning dirty laundry, the method comprising the steps of: (a) adding the dirty laundry to a solvent in the presence of a switchable surfactant, wherein the dirty laundry comprises one or more clothing items containing one or more stains caused by one or more staining agents; (b) causing the surfactant to enter an activated state, wherein the activated surfactant solubilizes at least one staining agent in the solvent; (c) physically separating the clothing items from the solvent; (d) causing the surfactant to return to the deactivated state, wherein the deactivated surfactant is soluble in the solvent but does not solubilize the staining agents; and (e) physically separating the staining agents from the solvent.

Still further disclosed herein is a method of osmotically purifying an impurity-containing source solution comprising the steps of: (a) providing a semipermeable membrane that permits contact between an osmotically-switchable purification solution and the source solution, wherein the osmotically-switchable solution comprises a solvent and a switchable surfactant capable of switching between activated and deactivated states, further wherein the switchable surfactant is soluble in the solvent in one state, and insoluble in the other state; (b) causing the switchable surfactant to enter its soluble state, wherein the resulting purification solution has a higher osmotic potential than the source solution; (c) isolating the purification solution; (d) causing the switchable surfactant to enter the insoluble state; and (e) physically separating the switchable surfactant from the purification solution.

### BRIEF DESCRIPTION OF THE FIGURES

FIG. 1 depicts a method of using a photoswitchable surfactant for thin-film or coating deposition.

FIG. 2 depicts a method of using a photoswitchable surfactant to extract a natural product from a source material.

FIG. 3 depicts a method of using a triggerable surfactant for a one-pot multi-step synthesis.

FIG. 4 is a schematic of how a photoswitchable surfactant may be used in a desalination process.

### DETAILED DESCRIPTION

While a number of exemplary embodiments, aspects and variations have been provided herein, those of skill in the art will recognize certain modifications, permutations, additions and combinations and certain sub-combinations of the embodiments, aspects and variations. It is intended that the following claims are interpreted to include all such modifications, permutations, additions and combinations and certain sub-combinations of the embodiments, aspects and variations are within their scope.

The entire disclosures of all documents cited throughout this application are incorporated herein by reference. The following procedures may be employed for the preparation of the compounds of the present invention. The starting materials and reagents used in preparing these compounds are either available from commercial suppliers such as the Aldrich Chemical Company (Milwaukee, Wis.), Bachem (Torrance, Calif.), Sigma (St. Louis, Mo.), or are prepared by methods well known to a person of ordinary skill in the art, following procedures described in such references as Fieser and Fieser's Reagents for Organic Synthesis, vols. 1-17, John Wiley and Sons, New York, N.Y., 1991; Rodd's Chemistry of Carbon Compounds, vols. 1-5 and supps., Elsevier Science Publishers, 1989; Organic Reactions, vols. 1-40, John Wiley and Sons, New York, N.Y., 1991; March J.: Advanced Organic Chemistry, 4th ed., John Wiley and Sons, New York, N.Y.; and Larock: Comprehensive Organic Transformations, VCH Publishers, New York, 1989.

### Definitions

Unless specifically noted otherwise herein, the definitions of the terms used are standard definitions used in the art of organic synthesis and chemical sciences. Exemplary embodiments, aspects and variations are illustrative in the figures and drawings, and it is intended that the embodiments, aspects and variations, and the figures and drawings disclosed herein are to be considered illustrative and not limiting.

While particular embodiments are shown and described herein, it will be obvious to those skilled in the art that such embodiments are provided by way of example only. Numerous variations, changes, and substitutions will now occur to those skilled in the art. It should be understood that various alternatives to the embodiments described herein may be employed in practicing the methods described herein. It is intended that the appended claims define the scope of the invention and that methods and structures within the scope of these claims and their equivalents be covered thereby.

Unless defined otherwise, all technical and scientific terms used herein have the same meaning as is commonly understood by one of skill in the art. All patents and publications referred to herein are incorporated by reference.

As used in the specification and claims, the singular form “a”, “an” and “the” include plural references unless the context clearly dictates otherwise.

An “alkyl” group, as used herein, refers to a straight, branched, saturated or unsaturated, aliphatic group having a chain of carbon atoms, optionally with oxygen, nitrogen or sulfur atoms inserted between the carbon atoms in the chain or as indicated. A (C₁₋₂₀ ) alkyl, for example, includes alkyl groups that have a chain of between 1 and 20 carbon atoms, and include, for example, the groups methyl, ethyl, propyl, isopropyl, vinyl, allyl, 1-propenyl, isopropenyl, ethynyl, 1-propynyl, 2-propynyl, 1,3-butadienyl, penta-1,3-dienyl, penta-1,4-dienyl, hexa-1,3-dienyl, hexa-1,3,5-trienyl, and the like.

An alkyl as noted with another group such as an aryl group, represented as “arylalkyl” for example, is intended to be a straight, branched, saturated or unsaturated aliphatic divalent group with the number of atoms indicated in the alkyl group (as in (C₁₋₂₀)alkyl, for example) and/or aryl group (as in (C₅₋₁₄)aryl or (C₆₋₁₄)aryl, for example) or when no atoms are indicated means a bond between the aryl and the alkyl group. Nonexclusive examples of such group include benzyl, phenethyl and the like.

An “alkylene” group is a straight, branched, saturated or unsaturated aliphatic divalent group with the number of atoms indicated in the alkyl group; for example, —(C₁₋₃)alkylene- or —(C₁₋₃) alkylenyl-.

A “cyclyl” such as a monocyclyl or polycyclyl group includes monocyclic, or linearly fused, angularly fused or bridged polycycloalkyl, or combinations thereof. Such cyclyl group is intended to include the heterocyclyl analogs. A cyclyl group may be saturated, partially saturated or aromatic.

“Halogen” or “halo” means fluorine, chlorine, bromine or iodine.

A “heterocyclyl” or “heterocycle” is a cycloalkyl wherein one or more of the atoms forming the ring is a heteroatom that is a N, O, or S. Non-exclusive examples of heterocyclyl include piperidyl, 4-morpholyl, 4-piperazinyl, pyrrolidinyl, 1,4-diazaperhydroepinyl, 1,3-dioxanyl, and the like.

“Substituted or unsubstituted” or “optionally substituted” means that a group such as, for example, alkyl, aryl, heterocyclyl, (C₁₋₈)cycloalkyl, heterocyclyl(C₁₋₈)alkyl, aryl(C₁₋₈)alkyl, heteroaryl, heteroaryl(C₁₋₈)alkyl, and the like, unless specifically noted otherwise, may be unsubstituted or, may substituted by 1, 2 or 3 substituents selected from the group such as halo, nitro, trifluoromethyl, trifluoromethoxy, methoxy, carboxy, —NH₂, —OH, —SH, —NHCH₃, N(CH₃)₂, —SMe, cyano and the like.

### Spiropyrans

In their ground state spiropyrans 1, are charge neutral, but when irradiated with the appropriate wavelength of light of energy hv (where h is Planck's constant and v is the frequency of the light corresponding to the appropriate wavelength) they undergo a ring opening photoreaction (“photochromic” behavior) to form the activated, zwitterionic merocyanine (MC) form, 2.

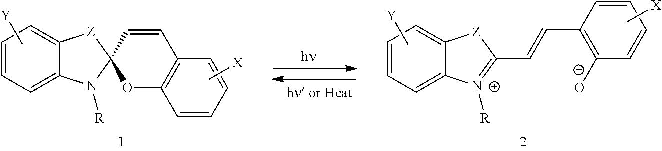

This spiropyran-merocyanine isomerism can also be triggered or “switched” in response to an electric field (electrochromic behavior), temperature (thermochromic behavior), pH, solvent polarity (solvatochromic behavior), or mechanical force.

The spiropyran-merocyanine interconversions are reversible, and either spontaneously under ambient thermal conditions, with removal of the electric field, with applied heat, and/or when irradiated by a different wavelength of light (hv′), the merocyanines revert to their original spiropyran form.

The physical properties of the spiropyran and merocyanine forms are very different. The ground state spiropyran is typically neutral, nonpolar and colorless, with hv typically ultraviolet light in the above reaction. On the other hand, the activated merocyanine is zwitterionic, polar and often highly colored, with hv′ in the reaction typically visible light.

The various properties of the spiropyran molecules 1 can be tailored by synthetically altering the X, Y, Z, and R substituents and the substitution pattern around the molecule. In particular, a spiropyran functional group can be derivatized so that the photochromic spiropyran moiety forms the head group of a surfactant molecule that can be reversibly switched between a de-activated, non-polar state and an activated, zwitterionic state. Since merocyanines spontaneously revert to the spiropyran configuration under ambient thermal conditions, simply removing the activating trigger—such as ultraviolet irradiation—can often deactivate the surfactant.

The tail functionality of the surfactant can be added at any (or all) of the substitution points X, Y, Z or R of the spiropyran. The R— substituent will most commonly be the substituent that functions as the non-polar tail of the surfactant molecule. The hydrophobic tail itself is not switchable. Suitable functional groups to form the hydrophobic tail include linear, long-chain alkyls such as n-octadecyl (C₁₈H₃₇—) or n-docosyl (C₂₂H₄₅—). Other common hydrophobic tails include alkyl-aryl substituents, branched hydrocarbons, ethers, amines, esters, or other linkages that do not disrupt the balance of polarity between the head and tail functionalities.

The R, X, Y, or Z substituent may also be a linker group that covalently attaches the switchable functional group to a polymer or polymeric backbone that comprises the hydrophobic tail portion of the surfactant, resulting in a photoactive polymeric surfactant, such as 3.

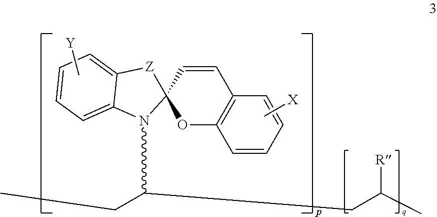

[symbol] is a linking group, R″ is a substituent on the polymer, p is the number of units of the polymer to which the spiropyran is covalently linked, and q is the number of units of the polymer that contain the R″ group. The p and q units may appear in any order—the above formula is only intended to define the relative proportion of monomer units and not the exact order.

In one aspect, the reversibly-switchable surfactant is of the formula I:

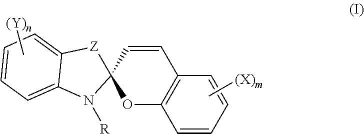

wherein:

m is 0, 1, 2, 3 or 4;

n is 0, 1, 2, 3 or 4;

R is H or is selected from the group consisting of substituted or unsubstituted C₁₋₂₂ alkyl, substituted or unsubstituted C₆₋₁₀ aryl, substituted or unsubstituted —C₁₋₂₂ alkyl-C₆₋₁₀ aryl, substituted or unsubstituted C₁₋₂₂ alkylC(O)—, substituted or unsubstituted C₁₋₂₂ alkylS(O)₁₋₂—, substituted or unsubstituted C₁₋₂₂ alkylNR′C(O)— and substituted or unsubstituted C₁₋₂₂ alkoxyC(NR″)—, or a polymer, bead, or resin;

each X is independently H or is selected from the group consisting of halo, —CN, —NO₂, —COOH, —SH, —OH, substituted or unsubstituted C₁₋alkyl, substituted or unsubstituted C₆₋₁₀ aryl, substituted or unsubstituted —C₁₋₂₂ alkyl-C₆₋₁₀ aryl, substituted or unsubstituted —C₁₋₂₂ alkylC(O)—, substituted or unsubstituted —C₁₋₂₂ alkylS(O)₁₋₂—, substituted or unsubstituted —C₁₋₂₂ alkylNR′C(O)— and substituted or unsubstituted C₁₋₂₂ alkoxyC(NR″)—, or a polymer;

each Y is independently H or is selected from the group consisting of halo, —CN, —NO₂, —COOH, —SH, —OH, substituted or unsubstituted C₁₋₂₂ alkyl, substituted or unsubstituted C₅₋₁₀ aryl, substituted or unsubstituted —C₁₋₂₂ alkyl-C₆₋₁₀ aryl, substituted or unsubstituted C₁₋₂₂ alkylC(O)—, substituted or unsubstituted C₁₋₂₂ alkylS(O)₁₋₂—, substituted or unsubstituted C₁₋₂₂ alkylNR′C(O)— and substituted or unsubstituted C₁₋₂₂ alkoxyC(NR″)—, or a polymer;

Z is —O—,—S—, —C(O)—, —C(S)—, —C(NR′)—, —S(O)₁₋₂—, —NR′— and —CR′R″—; and

R′ and R″ are each independently selected from the group consisting of H, substituted or unsubstituted C₁₋₆ alkyl and substituted and unsubstituted —C₁₋₆ alkyl-C₆₋₁₀ aryl.

In preferred embodiments of the above compound, X or Y is a n—C₁₈H₃₇ or n—C₂₂H₄₅ group. In another embodiment, one of X or Y is a C₈₋₂₂ n-alkyl group.

In another specific embodiment, the compound is 3,3-dimethyl-6′nitrospiropyran-1-propanoic acid or 3,3-dimethyl-6′nitrospiropyran-l-propanoic acid potassium salt:

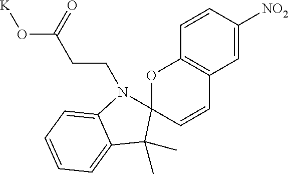

In some embodiments, R is a bead or resin. Beads or resins useful in the applications herein are well-known in the art.

In a different embodiment of the above, R comprises a linking group that covalently attaches the compound to a polymer or polymer backbone, resulting in the compound being a part of a polymer, such as the polymer compound of the formula II:

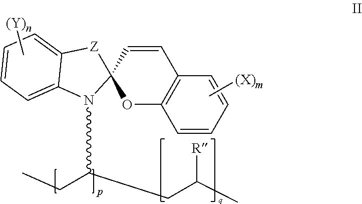

wherein:

[symbol] comprises a linking group,

m is 0, 1, 2, 3 or 4;

n is 0, 1, 2, 3 or 4;

each X is independently H or is selected from the group consisting of halo, —CN, —NO₂, —COOH, —SH, —OH, substituted or unsubstituted C₁₋₂₂ alkyl, substituted or unsubstituted C₆₋₁₀ aryl, substituted or unsubstituted —C₁₋₂₂ alkyl-C₆₋₁₀ aryl, substituted or unsubstituted —C₁₋₂₂ alkylC(O)—, substituted or unsubstituted —C₁₋₂₂ alkylS(O)₁₋₂—, substituted or unsubstituted —C₁₋₂₂ alkylNR′C(O)— and substituted or unsubstituted C₁₋₂₂ alkoxyC(NR″)—;

each Y is independently H or is selected from the group consisting of halo, —CN, —NO₂, —COOH, —SH, —OH, substituted or unsubstituted C₁₋₂₂ alkyl, substituted or unsubstituted C₅₋₁₀ aryl, substituted or unsubstituted —C₁₋₂₂ alkyl-C₆₋₁₀ aryl, substituted or unsubstituted C₁₋₂₂ alkylC(O)—, substituted or unsubstituted C₁₋₂₂ alkylS(O)₁₋₂—, substituted or unsubstituted C₁₋₂₂ alkylNR′C(O)— and substituted or unsubstituted C₁₋₂₂ alkoxyC(NR″)—;

Z is —O—, —S—, —C(O)—, —C(S)—, —C(NR′)—, —S(O)₁₋₂—, —NR′— and —CR′R″—;

R′ and R″ are each independently selected from the group consisting of H, substituted or unsubstituted C₁₋₆ alkyl and substituted and unsubstituted —C₁₋₆ alkyl-C₆₋₁₀ aryl;

R″′ is a substituent on the polymer, p is 0 or 1 to 100; and q is 0 or 1 to 100.

In another embodiment of the compound of formula I, X or Y is a n—C₁₈H₃₇ or n—C₂₂H₄₅ group. In another embodiment, one of X or Y is a C₈₋₂₂ n-alkyl group.

In another aspect, the molecular weight of the polymer is 1 to about 10 kilodaltons. In some embodiments, the molecular weight of the polymer is 400-600. In one aspect, R′″ is selected from the group consisting of —OC(O)CH₃, —OH, —OMe, —C₁₋₆ alkyl, —C₆H₅, —CF₃, —OCF₃ and —C(O)CH₃, or a mixture thereof. In another aspect, the polymer is attached to the nitrogen atom (N) of the compound by a group selected from —C₁₋₂₂ alkyl—, —C₆₋₁₀ aryl—, —C₁₋₆ alkyl-C₆₋₁₀ aryl—, —C₁₋₂₂ alkylC(O)—, —C(O)—, —C₁₋₂₂ alkylS(O)₁₋₂—, —C₁₋₂₂ alkylNR′C(O)—, —C₁₋₂₂ alkoxyC(NR′)—, —NR′C(O)— and —C(NR″)—, or a combination thereof.

In one embodiment, each polymer of formula II has a polymer backbone independently selected from the group consisting of polyethylene (LDPE or HDPE), polyethylene glycol (PEG), polypropylene, poly(vinyl chloride), poly(vinylidene chloride), polystyrene, polyacrylonitrile, polytetrafluoroethylene (PTFE, Teflon), poly(methyl methacrylate) (PMMA), poly(vinyl acetate) (PVAc), polyisoprene, polychloroprene, poly(oxyethylene) (POE), poly(oxy-1,2-ethanediyloxycarbonyl-1,4-phenylenecarbony (PET), poly[amino(1-oxo-1,6-hexanediyl)], polystyrene, ethyl-vinyl-acetate (EVA), polylactide (PLA), polyhydroxyalkanoates (PHA), polyhydroxybutyrate (PHB), poly-L-lactide (PLLA), PDLA (poly-D-lactide) or mixtures thereof. In some embodiments, the polymer backbone is PEG. In some of those embodiments, the molecular weight of the PEG is between 400 and 600.

The properties of the polymer II can also be altered by variation of the linking group attaching the compound to the polymer backbone. In one aspect, the properties of the polymer II can also be altered by variation of the R″′ substituent selected from acetate (—CO₂CH₃) or phenyl (—C₆H₅).

The properties of the polymer 3 can be altered by variation of the p:q ratio of the units that comprise the polymer. The value of q may be zero, in which case the polymer consists only of units functionalized by the reversibly-switchable head group 1 and no R″ groups are present.

The properties of the polymer 3 can also be altered by variation of the molecular weight of the polymer, that is, the chain length, which is determined by the total number of p and q units in each polymer chain.

The properties of the polymer 3 can also be altered by variation of the linking group attaching the compound to the polymer backbone. The linking groups can be selected to adjust the geometric relationship to the backbone, where the relationship includes distance and angle between the spiropyran moiety and the polymer backbone, and rigidity of the link, or other properties.

The properties of the polymer 3 can also be altered by variation of the R″ substituent of the polymer 3, with examples such as methacrylate (R″═acetate, —CO₂CH₃) or styrene (R″═phenyl, —C₆H₅) or other co-polymerizable moiety that does not alter the polarity balance between the head and tail of the polymer surfactant.

The properties of the polymer 3 can also be altered by preparing the polymer 3 as a “block” polymer. In the block polymer, discrete segments or blocks of the polymer are made by first polymerizing one monomer, and then subsequently a second block is added to the end of the first by polymerization of a second monomer; the sequence is repeated to synthesize a block polymer of the desired molecular weight.

The properties of the polymer 3 can also be altered by co-polymerization with yet another co-monomer that imparts other functionalities. Examples of other co-monomers include a crosslinkable moiety such as the photocrosslinker vinylbenzylthymine, or a divinyl monomer that polymerizes to form branched-chain polymers.

In one form of the reversible surfactant 1 there are the 1′-alkyl-3′,3′-dimethyl-6-nitrospiro[chromene-2,2′-indolines], that are the alkyl derivatives of the 6-nitrospirobenzopyran molecule 4, and its merocyanine isomer 5.

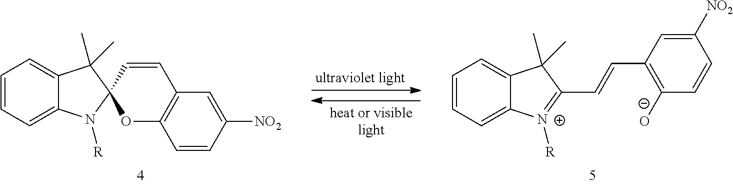

The π-acceptor ability of the nitro group enables delocalization of the negative charge on the phenolic oxygen in the ring-opened merocyanine, as seen in these resonance forms:

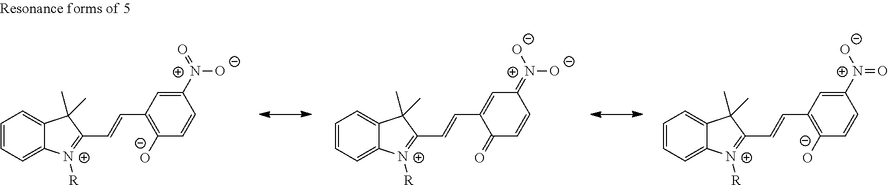

The resonance delocalization extends the conjugation of the π electrons, resulting in the absorption maxima, λₘₐₓ, of the nitro-merocyanines lying in the green to red portion of the visible spectrum, 500-600 nm. The nitro-merocyanines are highly solvatochromic, so any particular nitro-merocyanine often has a large enough shift in λₘₐₓ that it will appear a different color in different solvents.

These features of the nitro-spiropyrans/nitro-merocyanines make them suitable as reversibly-switchable surfactants. The lifetimes of the activated nitro-merocyanines are long enough in many solvents for the activated zwitterion to have time to function as a surfactant. The λₘₐₓ of the nitro-spiropyrans is in the ultraviolet, and the λₘₐₓ of the nitro-merocyanines in the visible, making the photon wavelengths used to photoswitch from spiropyran to merocyanine well separated from one another. Although not necessary for their use as surfactants, the solvatochromism makes the merocyanine distribution in multi-phase systems readily and visually apparent.

More specifically, spiropyran-based reversibly-switchable surfactants are disclosed that are exemplified by the tailed 6-nitro-spiropyrans 4a-f, each of which has a merocyanine isomer 5a-f.

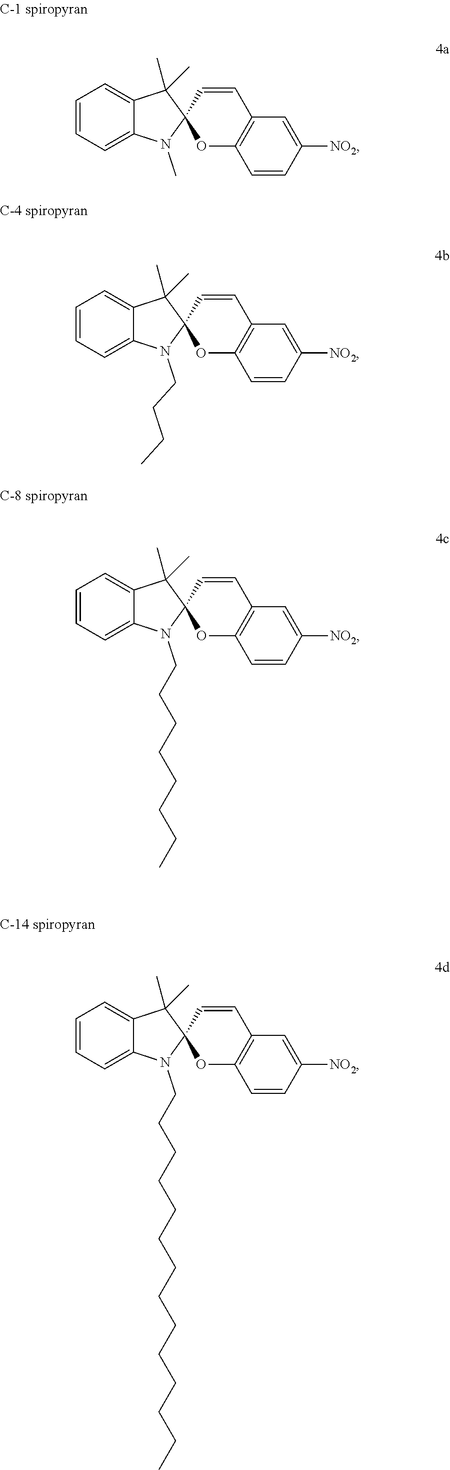 [SMILES: CN1c2ccccc2C(C)(C)[C@]12C=Cc1cc([N+](=O)[O-])ccc1O2 ; CCCCN1c2ccccc2C(C)(C)[C@]12C=Cc1cc([N+](=O)[O-])ccc1O2 ; CCCCCCCCN1c2ccccc2C(C)(C)[C@]12C=Cc1cc([N+](=O)[O-])ccc1O2 ; CCCCCCCCCCCCCCN1c2ccccc2C(C)(C)[C@]12C=Cc1cc([N+](=O)[O-])ccc1O2]

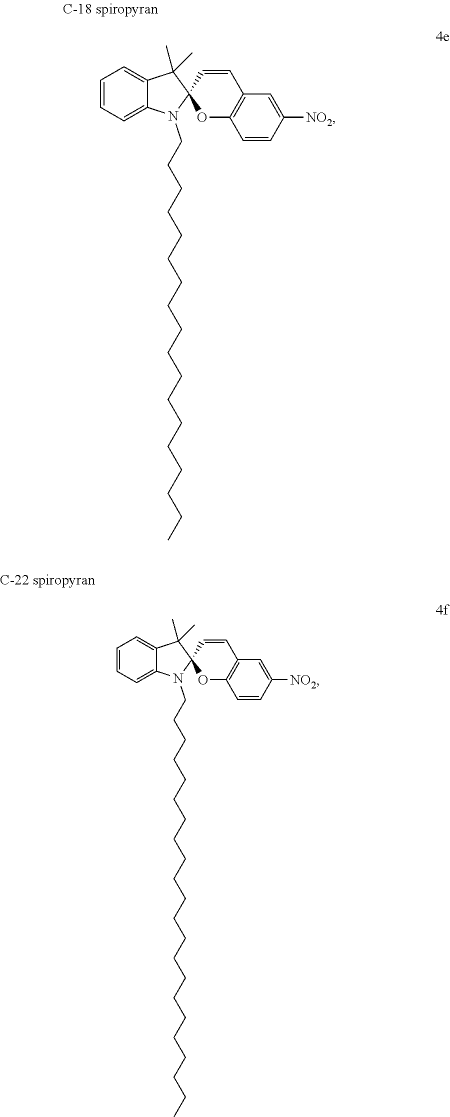 [SMILES: CCCCCCCCCCCCCCCCCCN1c2ccccc2C(C)(C)[C@]12C=Cc1cc([N+](=O)[O-])ccc1O2 ; CCCCCCCCCCCCCCCCCCCCCCN1c2ccccc2C(C)(C)[C@]12C=Cc1cc([N+](=O)[O-])ccc1O2]

### Methods of Using Switchable Surfactants

### A. Switchable Surfactants for Use in the Deposition of Thin Films and Surface Coatings.

In the semiconductor industry, thin films of polymers are used as photoresists, and layered polymeric thin-film structures make up the components of organic light-emitting diodes (OLEDs). Thin polymer films are typically produced by spin-coating of appropriate polymer and solvent mixtures onto a wafer substrate. Photovoltaics, including polymer and organic solar cells, dye-sensitized solar cells, and flexible batteries, are also composed of thin-film structures applied to a substrate. The large-scale production of thin-film structures for applications such as photovoltaics, printed electronics, or photographic film is generally accomplished by roll-to-roll (web) processing. Roll-to-roll processing is also used for less sophisticated applications, such as the manufacture of adhesive tape.

Production of a polymeric thin film for these and other applications requires coating a surface with the polymer dissolved in a solvent, whether by spin-coating or roll-to-roll processing including, but not limited to techniques such gravure printing, screen printing, slot coating, dip coating, or other methods. This is followed by evaporation of the solvent and the accompanying deposition of the thin-film material onto the substrate.

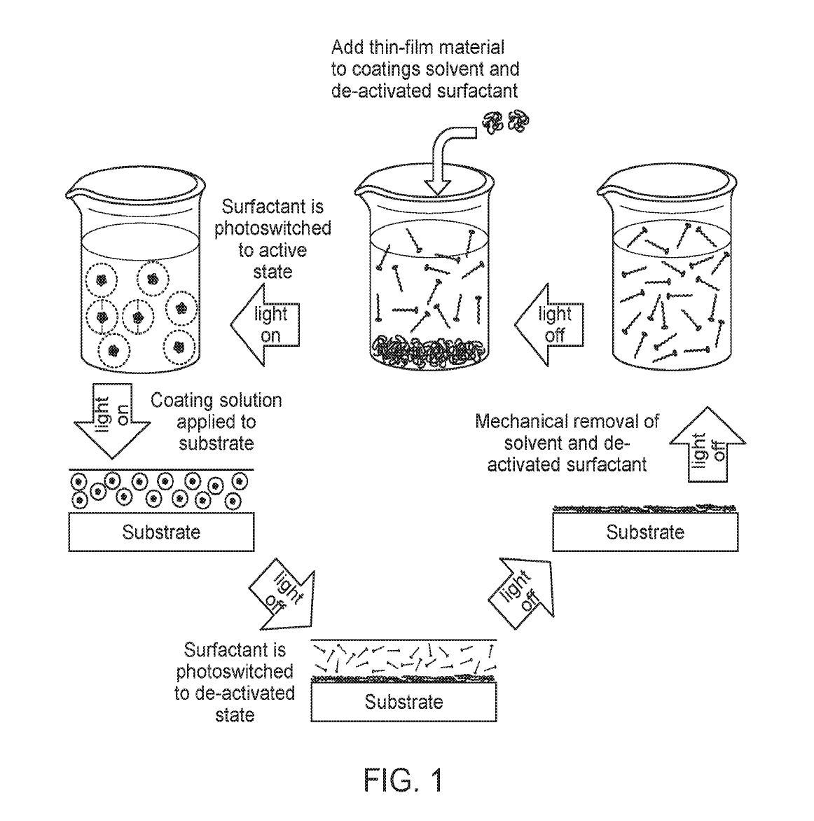

FIG. 1 illustrates one system whereby the solubility of the thin-film material can be switched between soluble and insoluble in the coating solvent by the use of a triggered switchable surfactant. Thin-film deposition is accomplished by activating the surfactant so as to dissolve the thin-film material in the solvent, coating the resulting solution, de-activating the surfactant so as to cause deposition of the now-insoluble thin-film material onto the substrate. The solvent and dissolved, de-activated surfactant is mechanically removed from the deposited thin film, such as by spinning the substrate (centrifugal force), aspiration, or by tilting and allowing the solvent and surfactant to drain from the deposited thin film.

The use of such a switchable surfactant enables deposition of the thin-film material without the need for evaporation of the solvent, eliminating hazards of solvent vapors and also greatly reducing energy costs. These reversible, switchable surfactants remain as intact molecules throughout the activated and de-activated forms, and the de-activated surfactant remains soluble in the solvent, so that the surfactant molecules do not contaminate the deposited thin-film material.

### B. Switchable Surfactants for Use in the Extraction of Natural Products.

Extraction and isolation of a natural product from a source material is a common commercial industrial process. Pharmaceuticals isolated from plants and fungi include the anti-cancer drug paclitaxel (taxol) which is isolated from bark of the Pacific Yew tree, morphine and other opiate alkaloids from opium poppies, the anti-cancer drugs vinblastine and vincristine from certain fungi, and the topical analgesic and anesthetic capsaicin from chili peppers. Flavors and fragrances, such as vanillin, cinnamaldehyde, linalool, lavender, sandalwood, or other essential oils, are derived from plants. Removal of caffeine from coffee beans is a case in which the decaffeinated beans, rather than the isolated caffeine itself, is the desired product, although the isolated caffeine is used as an ingredient in soft drinks and “energy” pills.

In a typical procedure to isolate the natural product from the source material, the plant (or fungal or animal) material is suspended in water. An organic solvent is added to the water suspension, and the desired natural product extracted into the organic solvent. The organic solvent and dissolved natural product is separated from the water dispersion, dried, and the solvent evaporated to yield the crude product. Isolation of the natural product by extraction into an organic solvent suffers from a number of drawbacks: The organic solvent may be toxic, and even small residues (or the threat of a residue) in the isolated product is undesirable; the cost of the volumes of organic solvent can be prohibitive, particularly if complete recovery requires multiple extraction steps; the removal of the organic solvent by evaporation from the natural product is energy-intensive and therefore adds to the overall process costs; the organic solvent can rarely be re-cycled and reused, so disposal costs and environmental hazards add to the overall cost of the process; water used in the extraction is contaminated by organic solvent, so disposal costs and environmental hazards add to the overall cost of the process.

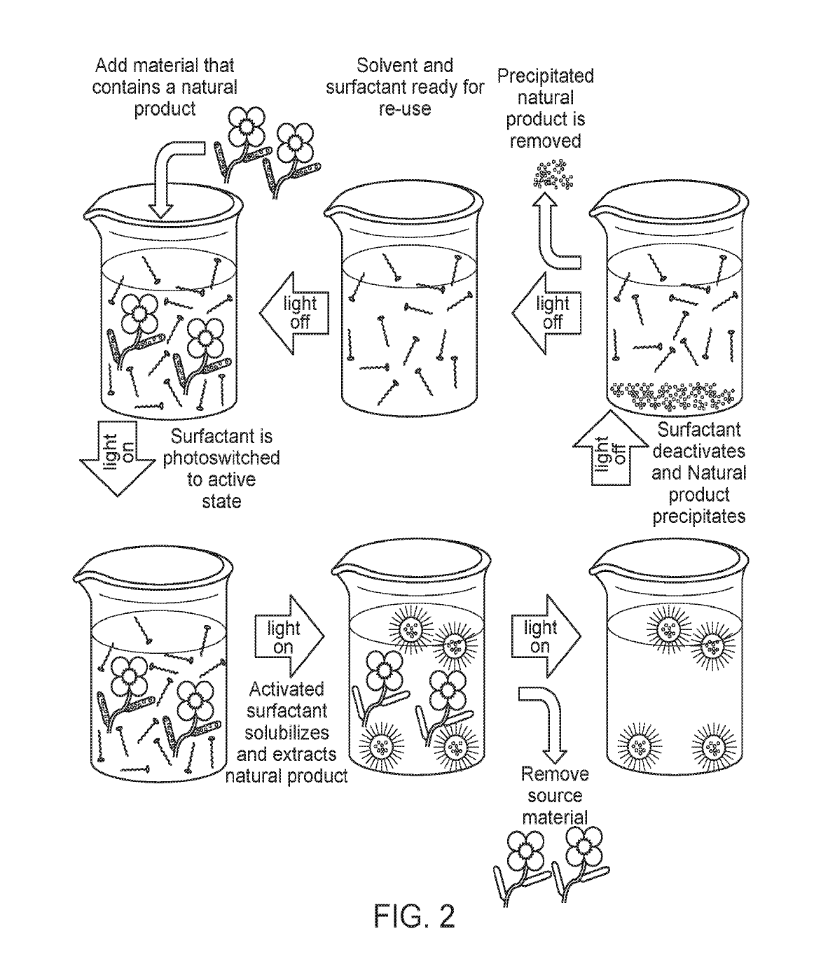

FIG. 2 illustrates the process of extracting and isolating a desired natural product from source material using switchable surfactants. A switchable surfactant and dispersed source material (such as a plant, fungus or animal material) is added to a solvent, and the surfactant activated, for example, by application of light. The activated surfactant increases the solubility of the natural product in the extraction solvent. The rate of product extraction may be increased by grinding the source material into small particles prior to adding to the solvent, or by heating, stirring, or sonicating the resultant solvent/surfactant/source material dispersion. A physical method (such as filtration or centrifugation) is used to remove the source material from the natural product/activated surfactant/solvent solution. The surfactant is then switched to a de-activated state, for example, by turning off the light or by illumination with light of a different wavelength. The de-activated surfactant no longer solubilizes the natural product in the solvent, and therefore the natural product separates out as a solid precipitate or other distinct phase such as an oil or organic liquid. The de-activated surfactant itself is selected to remain soluble in the extraction solvent. The natural product is then removed from the de-activated surfactant/solvent solution by a physical method, such as filtration, decantation, centrifugation, the use of a separating funnel, or the like. The switchable surfactant and solvent can be cycled through repeated extraction procedures.

The method of natural product isolation shown in FIG. 2 has several advantages. The ability to cycle the solvent and surfactant greatly reduces materials and disposal costs. Switching the surfactant causes the natural product to phase separate, so that there are no energy costs for solvent evaporation for its isolation. Since the surfactant is not only switchable but is chosen to remain intact and soluble in the solvent in both spiropyran and merocyanine forms, it will not contaminate the natural product. Although the use of the switchable surfactant is general for any solvent, it is preferred that water is the extraction solvent, so that no toxic solvents are used in the process.

### C. Switchable Surfactants for Use in One-pot Multi-step Syntheses.

Chemical synthesis of a compound is most typically a multi-step process, in which intermediate compounds are synthesized, isolated, purified, and then the intermediates are used as the precursor compound in the subsequent step in the synthetic procedure. The isolation of each intermediate compound at the end of each step is often necessitated by the reaction of unconsumed precursor molecules or the side products from an earlier step subsequently reacting to produce an unwanted and/or difficult to remove product, or otherwise interfering with or altering the course of subsequent reaction steps.

At each step in a multi-step synthesis, the isolation of the intermediate compound results in increased time to complete the total synthesis to the desired product. The use of large volumes of solvents and other materials (such as chemical drying agents, filters, or the like) engenders increased costs for the synthetic procedure. Disposal of the solvents, side products, and other materials has an additional economic cost and environmental impact. Yields of any intermediate compound as measured before isolation are almost invariably less than 100%, and the isolation and purification of intermediates reduces the yield even further. Since total synthesis of many chemicals, in particular, total synthesis of large pharmacoactive molecules can require 9 or more synthetic steps, a tremendous loss in final yield of the desired product occurs even if only a very small reduction in yield accompanies each intermediate synthetic step of the total synthesis.

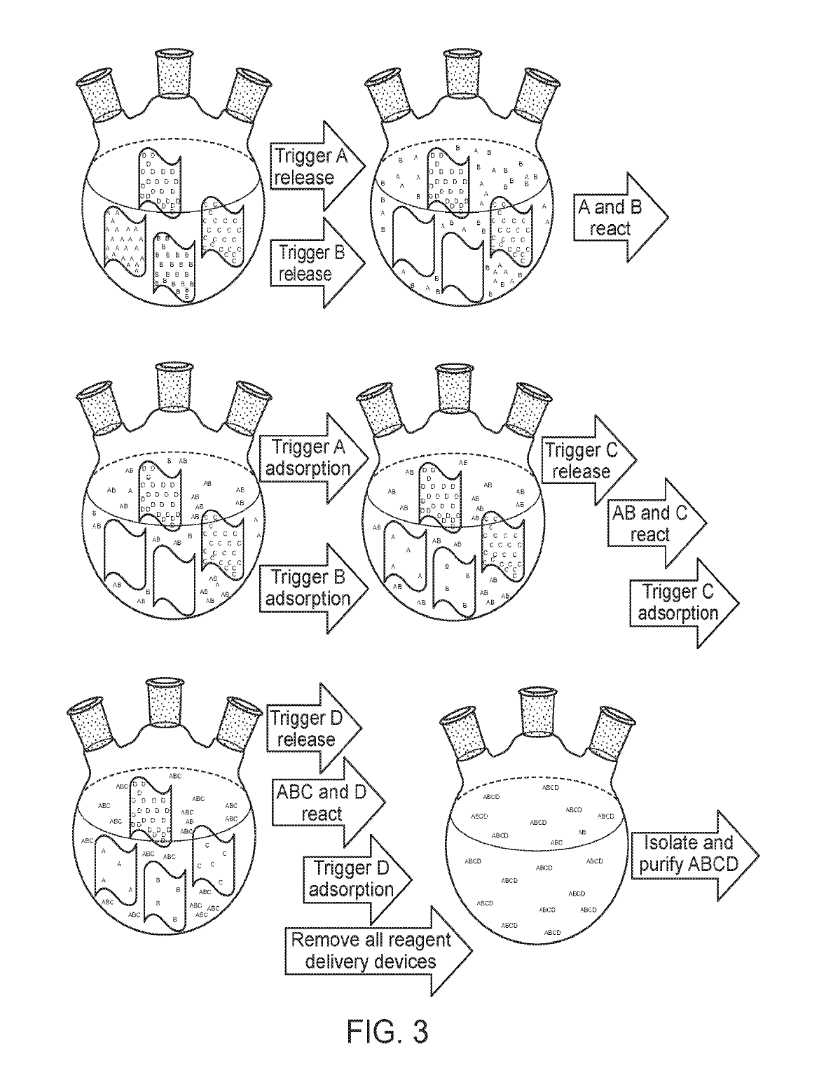

FIG. 3 illustrates a system that consists of a switchable surfactant and reagent-delivery device such as a sheet, bead, or granule that is a resin or an active coating on a solid support that is pre-loaded with an adsorbed reagent. The “reagent-delivery device” might also be comprised of a secondary phase solvent that contains the dissolved reagent. The surfactant and delivery device is added to a reaction vessel, and the surfactant activated to the form that solubilizes the pre-loaded reagent. After completion of the reaction step, the surfactant can be switched to the de-activated form, so the unconsumed reagent is re-adsorbed onto the delivery device.

In one method of use, the reagent-delivery devices may be added in sequence to the reaction vessel. Following re-adsorption of excess reagent at the end of each step, the reagent-delivery device is removed from the reaction vessel and the devices containing the reagents for the next step are added to the reaction vessel.

In another method of use, a complete set of individually triggerable surfactants and set of pre-loaded reagent-delivery devices can be added to a reaction vessel at the start of a multi-step synthesis. Each surfactant is triggered, resulting in solubilization of the appropriate reagent at the time it is needed in the series of reaction steps. Only at the end of the total synthesis will the devices be removed (or the secondary phase with dissolved reagents) and the product isolated from the reaction solvent. This method of use is the one illustrated in FIG. 3.

Whether or not the surfactants and reagent-delivery devices are added as a complete set at the start of the reaction or sequentially with each reaction step, the method enables the entire multi-step synthesis to be carried out in the single reaction vessel without isolation and purification of the intermediate compounds of the synthesis. The triggerable surfactants function as phase-transfer catalysts, facilitating the movement of any particular reagent both to and from an inaccessible secondary phase and the reaction solvent.

The switchable surfactants used to solubilize reagents A, B, C, D are not shown for clarity, but would be contained in the reaction solvent throughout the entire synthesis. Such a “one-pot multi-step” synthesis would substantially reduce the costs of materials, chemicals and their disposal; reduce energy usage; reduce the associated environmental impact of waste chemical disposal; reduce the time required to perform the synthesis; and improve yield of the final product.

### D. Switchable Surfactants for Use in the Extraction of Dirt and Stains (Laundering)

In the same way as the switchable surfactants of this invention can be used to extract a natural product from a source material, they can be used to extract dirt and stains from clothing—in other words, they can be used for laundering.

In the method of cleaning dirty laundry, the dirty laundry to be cleaned is added to a solvent (usually water) in the presence of a switchable surfactant. When the surfactant is activated, it will solubilize the dirt and stains, separating them into the solvent. The clothing items are then separated from the solvent. For example, in a conventional clothes washing machine, the aqueous solvent is drained from the machine while the clothing remains. The solvent is retained separately, and the surfactant is caused to return to its deactivated state. In this state, the surfactant remains in the solvent but does not solubilize the dirt and staining agents which then fall out of solution.

The dirt and staining agents are then physically separated from the solvent. The dirt and staining agents are discarded, while the surfactants may be recovered and recycled for further use.

### E. Switchable Surfactants for Use in Osmotic Processes

Integrating switchable surfactants into osmotic processes is another possibility. In a switchable surfactant based osmotic system, the switchable molecules are either integrated into an osmotic membrane or used as a drawing agent to remove an impurity or impurities from an impurity-containing solution. In one embodiment of this application, the impurity containing solution is seawater or saltwater, and the impurity is salt.

The osmotic membrane for this application consists of a hydrogel functionalized with switchable molecules, either on the surface of the membrane or integrated into the hydrogel matrix. Upon exposure to light, the switchable molecules change from hydrophobic form to hydrophilic form, drawing water into the membrane and separating it from contaminants. When the light is removed, the membrane deposits clean, extracted water into an alternate chamber and the process is repeated.

Alternatively, the switchable molecules could function as drawing agents, controllably increasing the osmotic pressure of the system. The switchable surfactants in this application could be small molecules or functionalized polymers. Upon exposure to light, the switchable molecules change from hydrophobic form to hydrophilic form, drawing clean water across the osmotic membrane. When the light is removed, the switchable molecules detach from the water molecules, and the process is repeated. In this embodiment the designed molecules or polymers are insoluble in hydrophobic state and soluble when molecules are in hydrophilic state. Water is separated from insoluble material by variety of techniques, including but not limited to pumping out, decanting, controlled close/open valves, etc.

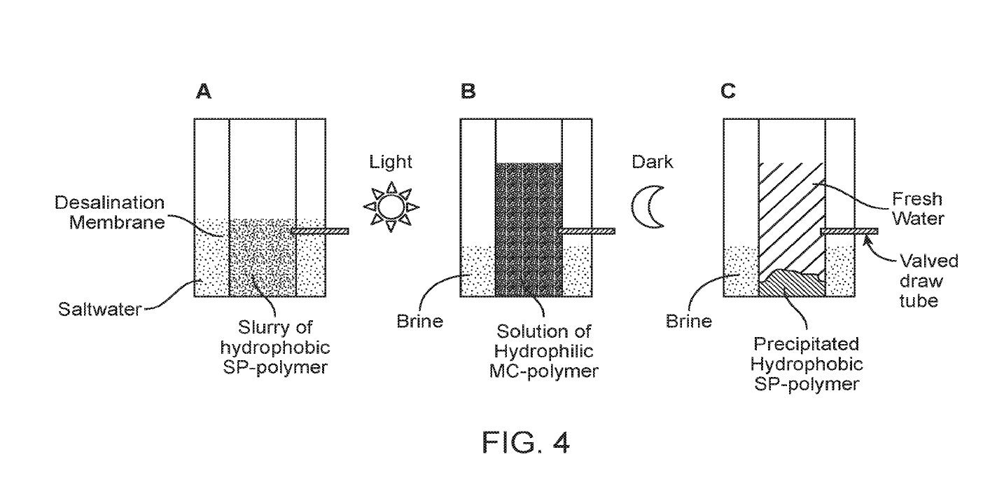

FIG. 4 depicts the use of a switchable material in a desalination process, using a cylinder within a cylinder, separated with a desalination membrane. At the start of the process (FIG. 4A), the inner chamber is filled with a slurry of hydrophobic switchable surfactant polymer. Seawater or saltwater is added to the outer chamber. The slurry is then activated with UVA light (either solar or artificial), which converts the hydrophobic surfactant (in this case a spiropyran polymer) to the hydrophilic form (in this case a merocyanine polymer). Since the polymer cannot cross the membrane, clean water is drawn out of the seawater into the inner chamber, leaving brine on the outside (FIG. 4B). Once the light is turned off, the hydrophilic polymer reverts to the hydrophobic form in an exothermic reaction, leaving behind fresh water that can be siphoned off (in this example, using a valved draw tube) and the process can be repeated (FIG. 4C).

One skilled in the art will recognize that for some switchable surfactants, the unactivated form may be hydrophilic while the activated form is hydrophobic. For those embodiments, the clean water is drawn into the inner chamber when the light is off, which is then siphoned off when the light is on.

F. Switchable Surfactants for Use in Fluid Delivery including Microfluidic devices

In this embodiment, switchable surfactants are integrated into a semipermeable membrane or a porous film for controlled fluid delivery. In a switchable surfactant-based fluid delivery system, either molecules are printed in a predesigned pattern or a light beam is used to make a pattern for fluid to pass through, depending on the architecture. The fluids may be aqueous or nonpolar solvents.

In one embodiment, switchable molecules or polymers are coated on a semipermeable membrane. In the hydrophobic state of the switchable molecules, the membrane pores allow only nonpolar solvents to get through, while in their hydrophilic state only polar solvents and water are allowed to pass. Depending on the desired application, stable hydrophilic or hydrophobic molecules are synthesized and switched to meta-stable orthogonal states by controlled light exposure.

In another embodiment, the switchable molecules are polymers coated on porous coatings made of nanoparticles (titanium oxide, silica, zirconia, etc.,), acting as a gate between feed solution and receiver solution. A light beam pattern converts a desired area into hydrophilic molecules allowing water based fluids (glucose solution, saline, blood, etc.) to move from feed to receiver sections. The gate is closed, upon switching off of light beam. Applications of these devices include, for example, controlled drug and insulin delivery, blood sampling, and microfluidic handling of explosive solutions.

### EXAMPLES

### Example 1

### Synthesis of C-1, 4, 8, 14, 18, and 22 Spiropyran

General synthesis of 1′-alkyl-6-nitrospiropyrans:

Spiropyran, 3′-dihydro-1′, 3′,3′-trimethyl-6-nitrospiro[2H-1-benzopyran-2,2′-(2H)-indole] is commercially available (TCI America, Portland, Oreg.). Other 1′-alkyl-6-nitrospiropyrans were synthesized by a 2-step procedure (Scheme 1, below) adapted from procedures reported in the literature for synthesis of various 1′substituted spiropyrans [Z. Tian et al., J. Am. Chem. Soc. 2011, 133(40), 16092-16100; D. Dattilo et al., Langmuir, 2007, 23 (26), pp 12945-12950].

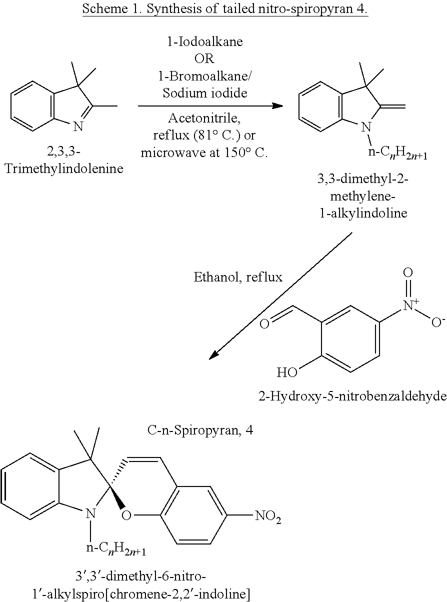

The first step in the synthesis is the addition of a linear alkyl group to 2,3,3-trimethylindolenine using an iodoalkane for the alkylating reagent. If the appropriate iodoalkane is not commercially available, the iodoalkane is generated in situ using a bromoalkane/sodium iodide combination. Typical reaction times in refluxing acetonitrile range from 17 hours to one or more days. Alternatively, the alkylation is achieved by microwave heating of the reagents in a sealed vial at 150° C. Microwave alkylation reduces the reaction times to 40-60 minutes, although the yields remain around 50% with a substantial portion of the starting trimethylindolenine remaining unalkylated.

The second step is the condensation of the alkylindoline with 2-hydroxy-5-nitrobenzaldehyde in refluxing ethanol. Reaction times are generally 15-24 hours and yields range from 37% to 85%. In all cases examined, the product 1′-alkylspiropyran precipitates out on cooling the reaction mixture, and is readily isolated by filtration and washing. In many instances the crystals can be used without further purification; flash chromatography can be used to improve purity of the tailed spiropyran.

All reagents were purchased from commercial suppliers and used as supplied unless stated otherwise. Reactions were carried out in air unless stated otherwise. 400 MHz 1H NMR spectra were obtained on a JEOL AS 400 spectrometer. A Teledyne ISCO ComiFlash® Rf200 UV/Vis chromatograph with autosampler was used for flash chromatography.

### Conventional Syntheses

Synthesis of 3′,3′-Dimethyl-6-nitro-1′-octadecylspiro[chromene-2,2′-indoline] (4e): 2,3,3-Trimethylindolenine (10.0 mL, 62 mmol, 1.0 equiv), sodium iodide (9.34 g, 62 mmol, 1.0 equiv) and 200 mL of acetonitrile was added to a 500-mL, three-necked, round bottom flask. The mixture was heated to reflux with stirring. Bromooctadecane (19.2 mL, 56 mmol, 0.9 equivalent) was added dropwise to the refluxing solution. The solution was refluxed for 7 days (158 hours), with occasional additions of acetonitrile to maintain solvent volume. The solvent was removed by evaporation with heating, and the residue was cooled to room temperature. The residue was extracted into acetonitrile, filtered and the filtrate concentrated under reduced pressure. The residue was dissolved in methylene chloride and transferred to a separatory funnel. The solution was washed with saturated sodium bicarbonate solution (2×) and water (1×). The organic phase was dried over anhydrous sodium sulfate and concentrated under reduced pressure, yielding 23.43 g of 3-dimethyl-2-methylene-1-octadecylindoline as an oil. (57 mmol, 92%).

5-Nitrosalicylaldehyde (6.71 g, 40 mmol, 1.0 equivalent) was added to 150 mL ethanol in a round bottom flask and brought to reflux. Crude 3-dimethyl-2-methylene-1-octadecylindoline (16.54 g, 40 mmol, 1.0 equivalent) was added dropwise to the refluxing solution. The mixture was refluxed overnight, cooled to room temperature and filtered. The collected solid was washed with ice-cold ethanol and dried under vacuum, to yield purple crystals of C-18-spiropyran (4e) (12.80 g, 23 mmol, 57%). ¹H NMR: (400 MHz, CDCl₃) δ 7.98 (m, 2 H), 7.16 (dd, 1 H, J=7.8, 7.7 Hz), 7.06 (d, 1 H, J=7.3 Hz), 6.87 (d, 1 H, 10.8 Hz), 6.84 (dd, 1 H, J=7.7, 7.3 Hz), 6.72 (d, 1 H, J=8.3 Hz), 6.55 (d, 1 H, J=7.8 Hz), 5.83 (d, 1 H, J=10.8 Hz), 3.11 (m, 2 H), 1.54 (m, 2H), 1.23 (m, 36 H), 0.86 (t, 3 H).

1′-Butyl-3′,3′-dimethyl-6-nitrospiro[chromene-2,2′-indoline], (4b): Pale yellow crystals of C-4 Spiropyran 4b were prepared similarly, using bromobutane/sodium iodide as the alkylating agent.

3′,3′-Dimethyl-6-nitro-1′-octylspiro[chromene-2,2′-indoline], (4c): Purple crystals of C-8 Spiropyran 4c were prepared similarly, using 1-iodooctane as the alkylating agent.

### Alternate Microwave Synthesis

1′-Docosyl-3′,3′-dimethyl-6-nitrospiro[chromene-2,2′-indoline], (4f). A 20 mL microwave vial containing a stirbar was charged with 2,3,3-trimethylindolenine (2.5 mL, 15.6 mmol, 1.0 equiv), 1-bromodocosane (7.894 g, 20.3 mmol, 1.30 equiv), sodium iodide (2.30 g, 15.3 mmol, 0.98 equiv) and 8 mL acetonitrile. The vial was placed in a Biotage® Initiator Classic microwave reactor and held at 150° C. for 40 minutes, and then cooled to room temperature. The contents of the vial were removed with ethyl acetate, added to a separatory funnel, and water added, resulting in an insoluble white solid. All layers were combined and the water and ethyl acetate removed under reduced pressure. The residue was dissolved methylene chloride, transferred to a separatory funnel and washed with water (1×) and then with 1 N NaOH (3×). The organic layer was dried over anhydrous sodium sulfate, filtered, and the filtrate concentrated under reduced pressure to yield dark red viscous liquid 1-docosyl-3,3-dimethyl-2-methyleneindoline (9.093 g).

5-Nitrosalicylaldehyde (1.437 g, 8.6 mmol, 1.0 equiv) was combined with 50 mL absolute ethanol and the mixture brought to reflux. 1-Docosyl-3,3-dimethyl-2-methyleneindoline (8.93 g of crude product containing an estimated 4.02 g of the tetradecylindoline, 8.6 mmol, 1.0 equiv) dissolved in 70 mL ethanol was added slowly to the refluxing solution. The reaction was refluxed for 19 hrs. After cooling to room temperature, the reaction mixture was filtered. The collected solids were washed with ice cold ethanol, and dried in a vacuum oven to yield a purple solid (7.1 g). The filtrate was concentrated under reduced pressure, and a second crop of purple solid collected by filtration, ethanol washing, and vacuum drying (1.61 g). The two crops of crystals were combined and purified by flash chromatography on silica with hexane/dichloromethane solvent, yielding 3.0 g (57%) of 4f. ¹H NMR (400 MHz, CDCl₃) δ 7.98 (m, 2 H), 7.16 (dd, 1H, J=7.8, 8.32 Hz), 7.06 (d, 1 H, J=7.3 Hz), 6.87 (d, 1 H, 10.8 Hz), 6.84 (dd, 1 H, J=7.3, 8.32 Hz), 6.72 (d, 1 H, J=8.8 Hz), 6.55 (d, 1H, J=7.8 Hz), 5.83 (d, 1 H, J=10.2 Hz), 3.10 (m, 2 H), 1.54 (m, 2H), 1.23 (m, 44 H), 0.85 (t, 3 H, J=6.8 Hz).

3′,3′-Dimethyl-6-nitro-1′-tetradecylspiro[chromene-2,2′-indoline], (4d). A yellow-orange powder of C-14 Spiropyran 4d was prepared similarly, using 1-bromotetradecane/sodium iodide as the alkylating agent.

3′,3′-Dimethyl-6-nitro-1′-octadecylspiro[chromene-2,2′-indoline], (4e): C-18 Spiropyran 4e was prepared similarly, using 1-bromooctabutane/sodium iodide as the alkylating agent. The product 4e was a beige powder with purple crystals mixed in.

### Example 2

### Lifetimes of the Activated Surfactants

An important criterion for the spiropyran/merocyanine switchable surfactants to be useful in a realistic setting is that the activated merocyanine form is long-lived enough to have a functional lifetime, e.g., to extract or solubilize a solute, or to form a micelle, in a solvent or solvents of interest.

Lifetimes of the activated zwitterionic merocyanine isomers of compounds 4a, 4b, 4c, and 4e in several solvents were determined.

The spiropyran compound 4 was added to a solvent in a 1-cm quartz cuvette and the cuvette was irradiated with ultraviolet light at 365 nm for ˜60 seconds. Irradiation resulted in a color change that indicates formation of the activated merocyanine. Absorption spectra were recorded from 450 to 650 nm every 5 seconds over a 350-second observation time. The concentrations of the merocyanine were adjusted so that the initial t=0 absorbance at the absorption maximum (λₘₐₓ) was in the range of 1-1.4. All kinetics experiments were done in triplicate once an appropriate spiropyran/merocyanine surfactant concentration was determined. The absorbances at λₘₐₓ were normalized to the highest value, and the three data sets averaged to give the normalized absorbances as a function of time. If the normalized absorbance had dropped to at least 0.5 during the 350-s monitoring time, the half-life was taken as the time at which the normalized absorbance had reached 0.5. If the half-life is longer than 350 s, the normalized absorbances were fit to a single exponential decay function and the half-life determined by extrapolation.

Half-lives for the nitro-merocyanines in all of the solvents studied are given in Table 1.

TABLE 1
Half-life of merocyanine surfactants in various solvents, in seconds. Error limits
are 10-20% of given values.

| | Merocyanine Half-life, in seconds | | | | | | |
|---|---|---|---|---|---|---|---|
| | Cyclo- | | Ethyl | | | | |
| merocyanine | hexane | Toluene | acetate | Acetone | Octanol | Ethanol | Methanol |
| C-1 merocyanine, | 14 | 16 | 19 | 103 | 610 | 1200 | 4900 |
| 5a | | | | | | | |
| C-4 merocyanine, | 44 | 32 | 23 | 130 | 610 | 1300 | 23000 |
| 5b | | | | | | | |
| C-8 merocyanine, | 40 | 38 | 20 | 153 | 610 | 1500 | >10⁴‡ |
| 5c | | | | | | | |
| C-18 merocyanine, | 40 | 49 | 20 | 114 | 830 | 1450 | 11000 |
| 5e | | | | | | | |

‡The lifetime of the C-8 merocyanine was so long that no decrease in absorbance was observed during the 350-second monitoring period.

Lifetimes of the nitro-merocyanines are in the range that makes them amenable to their use as a photo-switchable surfactant.

The strongest effect on lifetime is the presence of a hydrogen-bond donor solvent, such as octanol, ethanol and methanol, which can increase the half-lives of the nitro-merocyanines by an order of magnitude or more.

### Example 3

### Reversible Solubility of Compounds using Switchable Surfactants

To determine the change in solubility of a solvent in the presence of the activated surfactants, a 10 mL-sample of the colorless 1 mg/mL spiropyran solution was added to each of two vials each equipped with a stir bar. An initial sample of the solid solute was added to each vial, and additional smaller amounts of the solutes were added to each vial if the previously-added solute completely dissolved. If the added solute sample did not fully dissolve, one of the vials was then removed from the dark, and stirred next to an ultraviolet lamp operating at 365 nm. During UV exposure the solutions turned various shades of pink to red to purple, depending on the color of the merocyanine in the solvent. Additional solute was added to the UV-exposed vial, with stirring, until again some undissolved solute was observed in the vial, i.e., the solubility limit had been reached. At the same time, the same amount of additional solute was added to the vial kept in the dark, so as to confirm that the solute is not simply slow to solubilize.

Quantitative solubilities and their increases on surfactant activation for several spiropyran/merocyanine, solvent, and solute systems are given in Table 2. Errors on the solubilities (mg/mL) and the increase in solute solubility on surfactant activation are about 10-20% of given value.

The increase in solubility on surfactant activation ranges from a low of 2% increase for caffeine in acetone/C-18 spiropyran, to a high of 83% for palmitic acid in hexanes/C-8 spiropyran.

TABLE 2
Solubilities of Nicotinic acid, Caffeine and Palmitic acid solutes with and without
activation of C-8 and C-18 spiropyran surfactants, 4c and 4e, at surfactant concentration
1 mg/mL solvent.

| | | Solubilities | | | | | |
|---|---|---|---|---|---|---|---|
| | | Nicotinic acid | | Caffeine | | Palmitic acid | |
| | | in ground- | | in ground- | | in ground- | |
| | | state | increase on | state | increase on | state | increase on |
| | | spiropyran/ | surfactant | spiropyran/ | surfactant | spiropyran/ | surfactant |
| | Spiropyran/ | solvent | activation | solvent | activation | solvent | activation |
| Solvent | Merocyanine | (mg/mL) | (%) | (mg/mL) | (%) | (mg/mL) | (%) |
| Acetone | C-8, 4c | 1.5 | 94% | 9.6 | 15% | | |
| | C-18, 4e | 2.0 | 35% | 9.8 | 2% | | |
| Ethyl | C-8, 4c | | | 6.8 | 21% | | |
| Acetate | C-18, 4e | | | 6.3 | 15% | | |
| Hexanes | C-8, 4c | | | | | 36 | 83% |
| | C-18, 4e | | | | | 41 | 29% |

A separate experiment demonstrated the increased solubility of palmitic acid (hexadecanoic acid) in hexanes with switchable surfactant activation. It was first determined that the solubility of palmitic acid in hexanes is only 41 mg/mL. Each of two vials were charged with hexanes containing 1 mg/mL of the C-18 spiropyran 4e and 53 mg/mL of palmitic acid. The first vial was kept in the dark, so the C-18-spiropyran remained in the ground state; the solution remained colorless and the palmitic acid not fully dissolved, as evidenced by a white precipitate. The second vial was exposed to 365-nm ultraviolet light that activated the surfactant; the solution turned magenta due to the color of the merocyanine. The entire 53 mg/mL of palmitic acid dissolved, resulting in a completely clear solution with no remaining precipitate. When the UV-treated solution was placed in the dark, precipitate began to form, indicating reversibility of the surfactant to its deactivated form.

In summary, screening studies indicate a number of compounds can be solubilized by the activated merocyanine surfactants. The spiropyran-based surfactant can be cycled between active and de-activated form, with concomitant solubilization and precipitation of the solute.

As described herein, mechanistic interpretation is useful in order to illustrate the concepts and provide information on the various embodiments and aspects of the present application. However it is understood that the invention may also operate by a different mechanism or by a combination of various mechanisms not necessarily including the one described herein. Also under most circumstances it may not be possible to conclusively prove which mechanism is responsible for the observed effects. Therefore the provided above mechanistic interpretation is not to be interpreted as being either exclusive or limiting.

The foregoing examples of the related art and limitations are intended to be illustrative and not exclusive. Other limitations of the related art will become apparent to those of skill in the art upon a reading of the specification and a study of the drawings or figures as provided herein.

In addition to the exemplary embodiments, aspects and variations described above, further embodiments, aspects and variations will become apparent by reference to the drawings and figures and by examination of the following descriptions.

What is claimed is:

1. A photochromic, reversibly-switchable surfactant comprising the formula I: 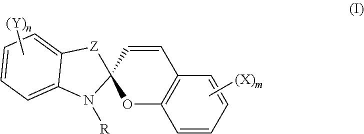
wherein:
m is 0, 1, 2, 3 or 4;
n is 0, 1, 2, 3 or 4;
R is H or is selected from the group consisting of substituted or unsubstituted C₁₋₂₂ alkyl, substituted or unsubstituted C₆₋₁₀ aryl, substituted or unsubstituted —C₁₋₆ alkyl-C₆₋₁₀ aryl, substituted or unsubstituted C₁₋₂₂ alkylC(O)—, substituted or unsubstituted C₁₋₂₂ alkylS(O)₁₋₂—, substituted or unsubstituted C₁₋₂₂ alkylNR′C(O)— and substituted or unsubstituted C₁₋₂₂ alkoxyC(NR″)—, or a polymer;
each X is independently H or is selected from the group consisting of halo, —CN, —NO₂, —COOH, —SH, —OH, substituted or unsubstituted C₁₋₆ alkyl, substituted or unsubstituted C₆₋₁₀ aryl, substituted or unsubstituted —C₁₋₆ alkyl-C₆₋₁₀ aryl, substituted or unsubstituted —C₁₋₆ alkylC(O)—, substituted or unsubstituted —C₁₋₆ alkylS(O)₁₋₂—, substituted or unsubstituted —C₁₋₆ alkylNR′C(O)— and substituted or unsubstituted C₁₋₆ alkoxyC(NR″)— or a polymer;
each Y is independently H or is selected from the group consisting of halo, —CN, —NO₂, —COOH, —SH, —OH, substituted or unsubstituted C₁₋₆ alkyl, substituted or unsubstituted C₅₋₁₀ aryl, substituted or unsubstituted —C₁₋₆ alkyl-C₆₋₁₀ aryl, substituted or unsubstituted C₁₋₆ alkylC(O)—, substituted or unsubstituted C₁₋₆ alkylS(O)₁₋₂, substituted or unsubstituted C₁₋₆ alkoxyC(NR″)— and substituted or unsubstituted C₁₋₆ alkoxyC(NR″)— or a polymer;
Z is —O—, —S—, —C(O)—, —C(S)—, —C(NR′)—, —S(O)₁₋₂—, —NR′— and —CR′R″—; and
R′ and R″ are each independently selected from the group consisting of H, substituted or unsubstituted C₁₋₆ alkyl and substituted and unsubstituted —C₁₋₆ alkyl-C₆₋₁₀ aryl.

2. The surfactant of claim 1, wherein R is n—C₁₈H₃₇ or n—C₂₂H₄₅.

3. The surfactant of claim 1, wherein at least one of X, Y or R comprises a C₁₋₂₂ alkyl group.

4. The surfactant of claim 1, wherein at least one of X, Y or R comprises a C₂₂ alkyl group.

5. The surfactant of claim 1, wherein at least one of X or Y comprises a C₂₂ alkyl group.

6. A photochromic, reversibly-switchable surfactant comprising the formula I: 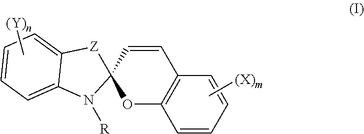
wherein:
m is 0, 1 or 2;
n is 0, 1 or 2;
R is H or is selected from the group consisting of substituted or unsubstituted C₁₋₂₂ alkyl, substituted or unsubstituted C₆₋₁₀ aryl, substituted or unsubstituted —C₁₋₂₂ alkyl-C₆₋₁₀ aryl, substituted or unsubstituted C₁₋₂₂ alkylC(O)—, substituted or unsubstituted C₁₋₂₂ alkylS(O)₁₋₂—, substituted or unsubstituted C₁₋₂₂ alkylNR′C(O)— and substituted or unsubstituted C₁₋₂₂ alkoxyC(NR″)—, or a polymer;
each X is independently H or is selected from the group consisting of halo, —CN, —NO₂, —COOH, —SH, —OH, substituted or unsubstituted C₁₋₂₂ alkyl, substituted or unsubstituted C₆₋₁₀ aryl, substituted or unsubstituted —C₁₋₂₂ alkyl-C₆₋₁₀ aryl, substituted or unsubstituted —C₁₋₂₂ alkylC(O)—, substituted or unsubstituted —C₁₋₂₂ alkylS(O)₁₋₂—, substituted or unsubstituted —C₁₋₂₂ alkylNR′C(O)— and substituted or unsubstituted C₁₋₂₂ alkoxyC(NR″)—, or a polymer;
each Y is independently H or is selected from the group consisting of halo, —CN, —NO₂, —COOH, —SH, —OH, substituted or unsubstituted C₁₋₂₂ alkyl, substituted or unsubstituted C₅₋₁₀ aryl, substituted or unsubstituted —C₁₋₂₂ alkyl-C₆₋₁₀ aryl, substituted or unsubstituted C₁₋₂₂ alkylC(O)—, substituted or unsubstituted C₁₋₂₂ alkylS(O)₁₋₂—, substituted or unsubstituted C₁₋₂₂ alkylNR′C(O)— and substituted or unsubstituted C₁₋₂₂ alkoxyC(NR″)—, or a polymer;
Z is —O—, —S—, —C(O)—, —C(S)—, —C(NR′)—, —S(O)₁₋₂—, —NR′— and —CR′R″—; and
R′ and R″ are each independently selected from the group consisting of H, substituted or unsubstituted C₁₋₆ alkyl and substituted and unsubstituted —C₁₋₆ alkyl-C₆₋₁₀ aryl.

7. A method of isolating a surfactant from a process employing the surfactant, the method comprising:
a) adding a deactivated surfactant to a process mixture to form the process mixture comprising the deactivated surfactant;
b) activating the surfactant to form the activated surfactant in the process mixture;
c) performing and completing the process to generate a process mixture comprising the activated surfactant and the desired product from the process; and
d) deactivating the surfactant to form a deactivated surfactant in the process mixture.

8. The process of claim 7 further comprising the step of isolating the deactivated surfactant from the process mixture.

9. The process of claim 7 further comprising isolating the desired product from the process mixture after step d) of deactivating the surfactant.

10. The process of claim 7 further comprising a recycling and re-use of the isolated deactivated surfactant.

11. The process of claim 7, wherein the activation of the surfactant is selected from the group consisting of applying light to the reaction mixture, generating an electric field to the reaction mixture, changing the reaction temperature, changing the pH of the reaction, changing the solvent polarity and applying a mechanical force to the reaction mixture, or combination thereof.

12. The process of claim 7, wherein the deactivation of the surfactant is performed by ultraviolet irradiation of the reaction mixture.

13. The process of claim 7, wherein the activation and deactivation of the surfactant is performed by a photochemical process.

14. A process for performing a surfactant mediated chemical reaction between a first reactant and a second reaction, the process comprising:
a) preparing a reaction mixture comprising the first reactant, the second reactant and the surfactant, wherein the surfactant is of the formula I: 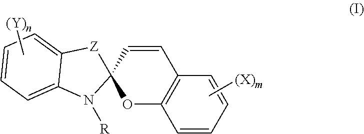
wherein:
m is 0, 1 or 2; n is 0, 1 or 2;
R is H or is selected from the group consisting of —C₁₋₂₂ alkyl, C₆₋₁₀ aryl, —C₁₋₂₂ alkyl-C₆₋₁₀ aryl, C₁₋₂₂ alkylC(O)—, C₁₋₂₂ alkylS(O)₁₋₂—, C₁₋₂₂ alkylNR′C(O)— and C₁₋₂₂ alkoxyC(NR″)—, or a polymer;
each X is independently H or is selected from the group consisting of halo, —CN, —NO₂, —COOH, —SH, —OH, —C₁₋₂₂ alkyl, C₆₋₁₀ aryl, —C₁₋₂₂ alkyl-C₆₋₁₀ aryl, —C₁₋₂₂ alkylC(O)—, C₁₋₂₂ alkylS(O)₁₋₂—, —C₁₋₂₂ alkylNR′C(O)— and C₁₋₂₂ alkoxyC(NR″)—, or a polymer;
each Y is independently H or is selected from the group consisting of halo, —CN, —NO₂, —COOH, —SH, —OH, —C₁₋₂₂ alkyl, C₅₋₁₀ aryl, —C₁₋₂₂ alkyl-C₆₋₁₀ aryl, C₁₋₂₂ alkylC(O)—, C₁₋₂₂ alkylS(O)₁₋₂—, C₁₋₂₂ alkylNR′C(O)— and C₁₋₂₂ alkoxyC(NR″)—, or a polymer;
Z is —O—,—S—, —C(O)—, —C(S)—, —C(NR′)—, —S(O)₁₋₂—, —NR′— and —CR′R″—; and
R′ and R″ are each independently selected from the group consisting of H, C₁₋₆ alkyl and —C₁₋₆ alkyl-C₆₋₁₀ aryl;
provided that at least one of X or Y comprises a C₂₂ alkyl group;
b) activating the surfactant to form the activated form of the surfactant to mediate the reaction between the first reactant and the second reactant; and
c) deactivating the surfactant to form the deactivated surfactant in the reaction mixture.

15. The process of claim 14, further comprising:
d) separating the deactivated surfactant from the reaction mixture.

16. The process of claim 14, wherein the compound of the formula I is:
m is 0 or 1; n is 0 or 1;
R is H or is selected from the group consisting of —C₂₂ alkyl, C₆₋₁₀ aryl, —C₂₂ alkyl-C₆₋₁₀ aryl, C₂₂ alkylC(O)—, C₂₂ alkylS(O)₁₋₂—, C₂₂ alkylNR′C(O)— and C₂₂ alkoxyC(NR″)—;
each X is independently H or is selected from the group consisting of halo, —CN, —NO₂, —COOH, —SH, —OH, —C₂₂ alkyl, C₆₋₁₀ aryl, —C₂₂ alkyl-C₆₋₁₀ aryl, —C₂₂ alkylC(O)—, —C₂₂ alkylS(O)₁₋₂—, —C₂₂ alkylNR′C(O)— and C₂₂ alkoxyC(NR″)—;
each Y is independently H or is selected from the group consisting of halo, —CN, —NO₂, —COOH, —SH, —OH, —C₂₂ alkyl, C₆₋₁₀ aryl, —C₂₂ alkyl-C₆₋₁₀ aryl, C₂₂ alkylC(O)—, C₂₂ alkylS(O)₁₋₂—, C₂₂ alkylNR′C(O)— and C₂₂ alkoxyC(NR″)—;
Z is —O—, —S—, —C(O)—, —C(S)—, —C(NR′)—, —S(O)₁₋₂—, —NR′— and —CR′R″—; and
R′ and R″ are each independently selected from the group consisting of H, C₁₋₆ alkyl and —C₁₋₆ alkyl-C₆₋₁₀ aryl;
provided that at least one of X or Y comprises a C₂₂ alkyl group.

17. A method of coating a surface with a thin-film material comprising the steps of:
(a) providing a solution of thin-film material in a solvent in the presence of a switchable surfactant
(b) causing the surfactant to enter the activated state, wherein the activated surfactant solubilizes the thin-film material:
(c) coating the surface with the solution;
(d) causing the surfactant to switch to the deactivated state, wherein the deactivated surfactant is soluble in the solvent but does not solubilize the thin-film material; and
(e) physically removing the solution from the surface.

18. A method of extracting a natural product from its source material, the method comprising the steps of:
(a) providing an admixture of source material in a solvent in the presence of a switchable surfactant;
(b) causing the surfactant to enter an activated state, wherein the activated surfactant solubilizes the natural product in the solvent:
(c) physically separating the source material from the solvent;
(d) causing the surfactant to return to the deactivated state, wherein the deactivated surfactant is soluble in the solvent but does not solubilize the natural product; and
(e) physically separating the natural product from the solvent.

19. A method of cleaning dirty laundry, the method comprising the steps of:
(a) adding the dirty laundry to a solvent in the presence of a switchable surfactant, wherein the dirty laundry comprises one or more clothing items containing one or more stains caused by one or more staining agents;
(b) causing the surfactant to enter an activated state, wherein the activated surfactant solubilizes at least one staining agent in the solvent:
(c) physically separating the clothing items from the solvent;
(d) causing the surfactant to return to the deactivated state, wherein the deactivated surfactant is soluble in the solvent but does not solubilize the staining agents; and
(e) physically separating the staining agents from the solvent.

20. A method of osmotically purifying an impurity-containing source solution comprising the steps of:
(a) providing a semipermeable membrane that permits contact between an osmotically-switchable purification solution and the source solution, wherein the osmotically-switchable solution comprises a solvent and a switchable surfactant capable of switching between activated and deactivated states, further wherein the switchable surfactant is soluble in the solvent in one state, and insoluble in the other state;
(b) causing the switchable surfactant to enter its soluble state, wherein the resulting purification solution has a higher osmotic potential than the source solution;
(c) isolating the purification solution;
(d) causing the switchable surfactant to enter the insoluble state; and
(e) physically separating the switchable surfactant from the purification solution.

21. The method of claim 20 wherein the impurity-containing source solution is seawater or saltwater and the impurity is salt.
